(19) United States
(12) Patent Application Publication
(10) Pub. No.: US 2009/0221987 A1
(43) Pub. Date: Sep. 3, 2009

(54) Portable sinus and throat cleansing system and method

(76) Inventor: Syeda J.Z. Mansur, Manassas, VA (US)

Correspondence Address: Cislo & Thomas LLP, 1333 2nd Street, Suite #500, Santa Monica, CA, 90401-4110, US

(21) Appl. No.: 12/072,970

(22) Filed: Feb. 29, 2008

(51) Int. Cl. A61M 3/02; A61M 37/00; A61M 31/00

(52) U.S. Cl. 604/514; 604/92; 604/416

(57) ABSTRACT

The present invention is generally directed to a system and method for cleansing mucus and in particular, removing mucus from the nasal cavity, mouth, and throat. The sinus and throat cleansing kit includes packets of dry saline, packets of dry salt, a spoon for mixing the dry saline and dry salt with water to produce a saline solution and salt solution, a pot for pouring the saline solution into a nostril of the user cleansing a nasal cavity of mucus, a container lid for pouring the salt solution into the mouth of the user cleansing a mouth and throat of mucus, and a container for collecting saline solution, salt solution, and mucus. The sinus and throat cleansing kit provides a clean and portable way to remove mucus, bacteria, and other viruses of the throat.

### FIELD OF THE INVENTION

This invention generally relates to providing relief, and more specifically, a portable sinus and throat cleansing system and method for removing mucus from the nostrils, mouth, and throat of a user.

### BACKGROUND OF THE INVENTION

A large number of individuals are afflicted with sinus conditions, respiratory allergies, and other discomforts of the nasal cavity and throat. These afflictions come from a variety of sources including viral infections, bacteria, air pollution, and the like. The nasal cavity, mouth, and throat are common places for holding these infectious agents that may cause allergic reactions and other ill consequences.

The most effective way to combat those infectious agents is to upkeep personal hygiene by irrigating the mucus from the nasal cavity and throat. Spray bottles devices have often been used for irrigating the mucus from the nasal cavity. Such devices, however, have several drawbacks. Many of these drawbacks are due to the fact that the user applies an unregulated force to squeeze the spray bottle, thus making the amount of fluid or dosage of spray administered to the nose not precisely determinable. Also, the uncontrolled force being applied would cause problems in damaging sensitive nasal membranes due to too much force being applied, or even a messy back flow of fluid and possibly nasal mucous onto the user. Furthermore, the spray bottle devices do not remove lingering mucus located within the mouth and throat.

### SUMMARY OF THE INVENTION

The present invention is generally directed to a system and method for providing relief to the nasal cavity, mouth, and throat. In accordance with one aspect of the present invention, a mucus cleansing kit is presented. The kit includes a container lid, at least one or more packets of dry saline, and a spoon for mixing the dry saline from the at least one or more packets with water to produce a saline solution. Also, the kit includes a pot for holding the saline solution, wherein the saline solution from the pot is poured into a nostril of a user cleansing a nasal cavity of mucus. Further, the kit includes at least one or more packets of dry salt. The spoon is used for mixing the dry salt from the at least one or more packets with water to produce a salt solution. The salt solution is poured into the mouth of the user cleansing a mouth and throat of mucus. The kit includes a container for collecting the saline solution, salt solution, and mucus.

In accordance with another aspect of the present invention, a method of cleansing mucus is presented. The method includes removing mucus from a nasal cavity by pouring a saline solution into a nostril of a user. The method also includes removing mucus from a mouth and throat by pouring a salt solution into a mouth of the user.

In accordance with yet another aspect of the present invention, a method for cleansing mucus with a kit is presented. The cleansing kit includes at least one or more packets of dry saline, a pot, a spoon, a container, and a container lid. The method includes mixing the at least one or more packets of dry saline and water with the spoon. Furthermore, the method includes inserting a spout of the pot into a first nostril of the user and rotating the head of the user so that the first nostril with the spout is above a second nostril. The method also includes placing the container under the second nostril and pouring the saline solution into the first nostril of the user, wherein the saline solution and the mucus empties out of the second nostril into the container.

### BRIEF DESCRIPTION OF DRAWINGS

The foregoing aspects and many of the attendant advantages of this invention will become more readily appreciated as the same become better understood by reference to the following description, when taken in conjunction with the accompanying drawings, wherein:

FIG. 1A illustrates an exemplary pot used for mixing and pouring solutions into a nostril of a user in accordance with one embodiment of the present invention;

FIG. 1B illustrates an exemplary side view of a pot for pouring solutions into a nostril of a user in accordance with one embodiment of the present invention;

FIG. 2A illustrates an exemplary container used for collecting solutions and other materials from a nostril in accordance with one embodiment of the present invention;

FIG. 2B illustrates an exemplary container lid used for mixing solutions in accordance with one embodiment of the present invention;

FIG. 3 illustrates an exemplary spoon used for mixing solutions in accordance with one embodiment of the present invention;

FIG. 4 illustrates exemplary pre-mixed saline and salt dry ingredient packages in accordance with one embodiment of the present invention;

FIG. 5 illustrates an exemplary portable sinus cleansing kit in accordance with one embodiment of the present invention;

FIG. 6A is an example of an initial step for using a portable sinus cleansing kit in accordance with one embodiment of the present invention; and

FIG. 6B is an example of the next step for using a portable sinus cleansing kit in accordance with one embodiment of the present invention.

### DETAILED DESCRIPTION OF THE PREFERRED EMBODIMENT

This invention generally relates to providing relief. More specifically, the invention is a portable sinus and throat cleansing system and method for removing unwanted bacteria from the nostrils, mouth, and throat of a user. The system and method provide the user with a clean way to remove mucus from the nostrils, mouth, and throat. Furthermore, the present invention is a self-enclosing kit capable of holding each item used to cleanse mucus allowing portability of the kit.

Those skilled in the relevant art would appreciate that the present invention is also applicable to other mucus producing membranes. The following first provides a description of each of the individual components of the kit. Next, the portable cleansing kit will be described. Then a description of the cleansing method will be described. The illustrative examples provided herein are not intended to be exhaustive or to limit the invention to the precise forms disclosed. Similarly, any steps described herein may be interchangeable with other steps or combinations of steps, in order to achieve the same result.

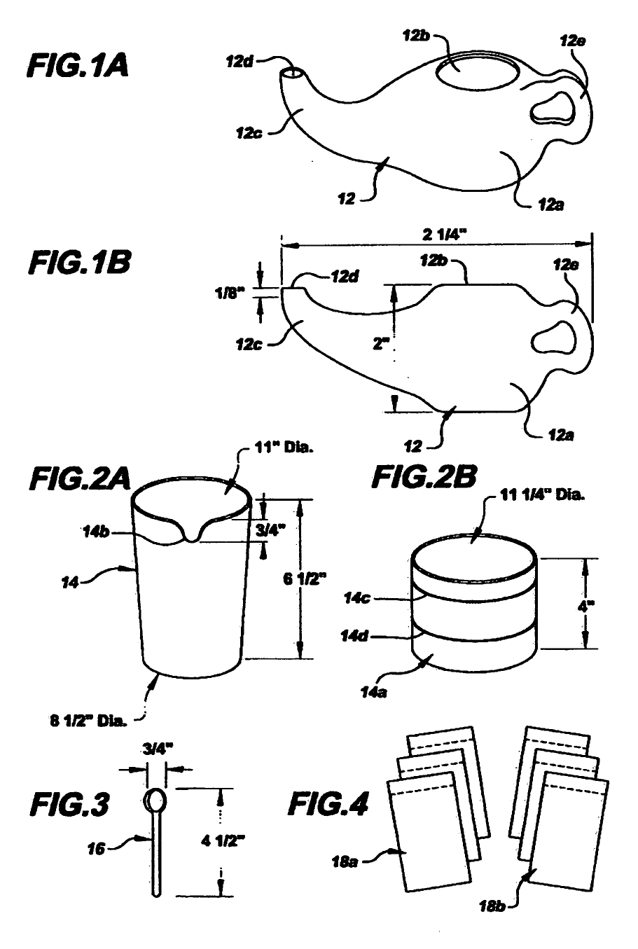

To provide a context for describing embodiments of the invention, the cleansing kit includes at least one or more packets of dry saline, a pot, a spoon, a container, and a container lid. FIG. 1A illustrates an exemplary pot 12 used for mixing and pouring solutions into a nostril of a user in accordance with one embodiment of the present invention. One skilled in the art would appreciate that the pot 12 is for illustrative purposes and may come in a wide variety of shapes and forms. In this embodiment, the pot 12 includes a main body 12a.

On the top portion of the pot 12 is an opening 12b. Generally, the opening 12b of the pot 12 includes a lid so that liquid will not spill out. In one embodiment of the present invention, the lid can tightly seal the opening 12b of the pot 12 in order to prevent any liquid or solution from coming out. Alternatively, the lid can be loosely attached to the opening 12b of the pot 12. In another embodiment, the pot 12 does not contain a lid to cover the opening 12b. In yet another embodiment, the lid allows liquids and other substances to enter through the opening 12b of the pot 12 without letting the liquid or substances exit though the opening 12b of the pot 12. One skilled in the art would appreciate that there are many different combinations for combining a lid to the opening 12b of the pot 12.

Continuing with FIG. 1A, the pot 12 also includes a spout 12c. Through the spout 12c of the pot 12, liquid or solution may funnel through the opening of the spout 12d. As will be described later, the opening of the spout 12d includes a portion that creates a tight seal between the user's nostril and the pot 12.

The pot 12 also contains a handle 12e. As shown, the handle includes a circular portion with a hole in it in order for the user to easily hold the pot 12. In one embodiment of the present invention, the handle 12e of the pot 12 includes texture giving the user better grip of the pot 12. In another embodiment of the present invention, the handle 12e of the pot 12 includes a rough material to give the user better grip of the pot 12.

FIG. 1B illustrates an exemplary side view of a pot 12 for pouring solutions into a nostril of a user in accordance with one embodiment of the present invention. Similar to FIG. 1A, the side view of the pot 12 in FIG. 1B includes a main body 12a, an opening 12b, a spout 12c, an opening of the spout 12d, and a handle 12e. In one embodiment of the invention, the pot 12 has a length of 2¼ inches, a height of 2 inches, and a spout opening 12d of ⅛ of an inch. This allows for the user to pour solutions within the pot 12 into the nose without having the spout 12c inserted too far into the nose. One skilled in the art would appreciate that the measurements can be changed depending on the user's preferences and their physical features.

With reference to FIG. 2A, an exemplary container 14 used for collecting solutions and other materials from a nostril in accordance with one embodiment of the present invention is presented. The container 14 includes a v-shaped recessed lip 14b. The v-shaped recessed lip 14b allows for collection of mucus and liquids. One skilled in the art would appreciate that there are different types of lips 14b that can be placed onto a container 14.

As shown in FIG. 2A, typical dimensions of the container 14 include 8½ inches for the diameter, 6½ inches for height, and ¾ inches from the top of the container 14 to the lip 14b of the container 14. One skilled in the art would appreciate that these dimensions are exemplary. Furthermore, one skilled in the art will appreciate that the container 14 may come in a variety of shapes and is not limited to a cylinder.

With reference to FIG. 2B, an exemplary container lid 14a used for mixing solutions in accordance with one embodiment of the present invention is presented. The container lid 14a can be attached to the container 14 through various ways. In one embodiment of the invention, the container lid 14a includes a set of corkscrew engravings on the inside, while the container 14 includes an opposite set of corkscrew engravings on the outside. When the container lid 14a is screwed onto the container 14, the container lid 14a and the container 14 create a tight seal and can hold items within it. In one embodiment, the items for cleansing the mucus are held within the container lid 14a and container 14. One skilled in the art would appreciate that there are many ways of attaching the container lid 14a to the container 14.

As shown in this embodiment, the container lid 14a has a height of 4 inches and has a diameter of 11¼ inches. Typically, the container lid 14a can come in many dimensions, forms, and shapes depending on the size of the items to be placed within the container 14 and the container lid 14a.

On the side of the container lid 14a are a number of measurement guides for displaying the amount of liquid or solution inside the container lid 14a. The container lid 14a includes an 8 oz. marker 14c printed on the outside of the container lid 14a. Typically, the 8 oz. marker 14c is used for making a salt solution. Further, the container lid 14a includes a 4 oz. marker 14d printed on the outside of the container lid 14a. Generally, the 4 oz. marker 14d is used for making the saline solution. Although only two markers 14c14d are shown, one skilled in the art would appreciate that there can be any number of markers indicating any amounts of liquid outside the container lid 14a.

FIG. 3 illustrates an exemplary spoon 16 used for mixing solutions in accordance with one embodiment of the present invention. In one embodiment of the present invention, the dimensions of the spoon 16 is ¾ inch wide by 4½ inches high. The spoon 16 can come in a wide variety of shapes and sizes. The spoon 16 can also be composed of different elements including metal, plastic, or the like.

With reference to FIG. 4, exemplary pre-mixed saline 18a and salt 18b dry ingredient packets in accordance with one embodiment of the present invention are presented. In one embodiment of the present invention, 40 pre-mixed saline dry ingredient packets 18a and 40 pre-mixed salt dry ingredient packets 18b are included. One skilled in the art, however, would appreciate that any number of saline 18a or salt 18b packets can be included within the present invention.

Typically, the active ingredients in each pack of saline 18a include 700 mg of sodium bicarbonate and 2300 mg of sodium chloride, while the active ingredients in each pack of salt 18b include 590 mg of sodium chloride and 2 mg of calcium silicate. Generally, no inactive ingredients exist either in the saline 18a or salt 18b packets.

When the dry saline 18a or salt 18b is mixed with water, the saline and salt solutions provides temporary relief of symptoms associated with soar throat, sinusitis, common cold, influenza, or allergies. The solutions also reduce sneezing, runny nose, nasal congestion, and post nasal drip. By breaking down the mucus, the solutions aid the nasal and sinus drainage. Furthermore, the solutions relieve sinus pressure and soothes soar throat by reducing the swelling of the throat and nasal membranes.

The process of using the salt and saline solutions is a drug free system that uses natural ingredients. The solutions aid in the body's natural defense in the battle against foreign elements entering into the body's respiratory system and lungs. Because of this reason, the solutions can be used as often as needed.

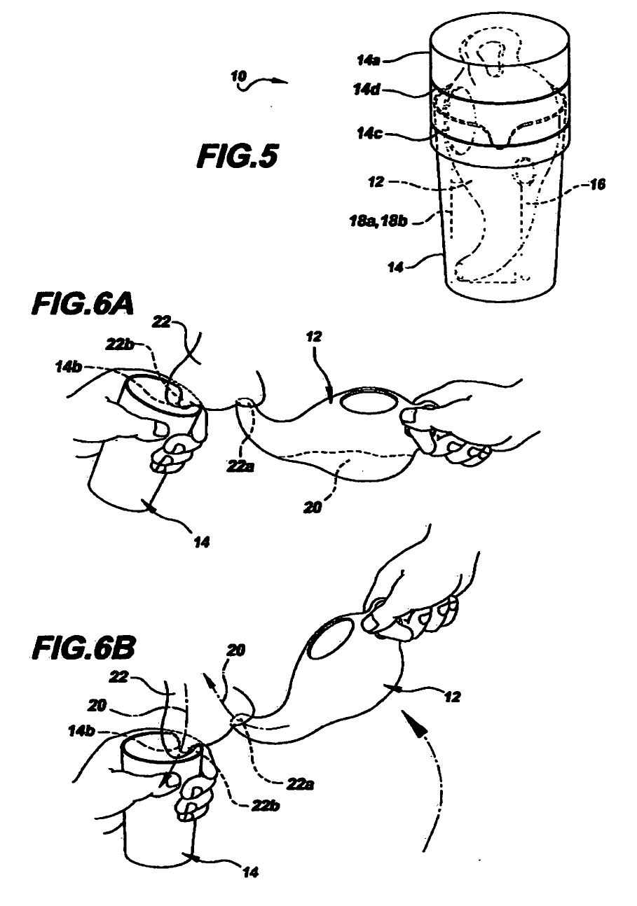

FIG. 5 illustrates an exemplary portable sinus cleansing kit 10 in accordance with one embodiment of the present invention. The container 14 and the container lid 14a house each of the above mentioned components of the kit 10 including the pot 12, the spoon 16, and the saline 18a and salt 18b packets. Because of the container 14 and container lid 14a packaging, the kit is portable and convenient for the user.

The method of using the portable sinus and throat cleansing kit 10 includes removing mucus from a nasal cavity by pouring a saline solution into a nostril of a user and removing mucus from a throat by pouring a salt solution into a mouth of the user. For a nasal cavity wash, the user empties the contents of one dry saline ingredient packet 18a into the pot 12. Typically, the user will start with ½ a packet 18a per pot 12. One skilled in the art would appreciate that the more saline packets 18a used, the higher the concentration of the saline.

In turn, the user fills the container lid 14a to a marked line on the lid 14a with lukewarm water depending on the amount of dry saline 18a placed into the pot 12 and the desired concentration. Typically, the user will add 4 oz. of water for every half packet of dry saline 18a used, which corresponds to the 4 oz. marker 14d on the container lid 14a. When the user empties a complete packet of dry saline 18a, 8 oz. of water should be used, which corresponds to the 8 oz marker 14c on the container lid 14a. The water should be lukewarm to dissolve the dry saline 18a. Alternatively, the pot 12 can be heated with the water inside. Further, the container lid 14a can be microwave safe so that the water can be heated in the microwave. One skilled in the art would appreciate that there are many ways to heat the water.

The user then stirs or mixes the water with the spoon 16 to dissolve the dry saline 18a. The user continues to stir thoroughly until the dry saline 18a has completely dissolved.

With reference to FIG. 6A, an initial step for using a portable sinus cleansing kit 10 in accordance with one embodiment of the present invention is presented. As shown, the user leans over their head with their nose 22 bent down. The user then holds the pot 12 in their left hand and gently inserts the end of the spout 12c of the pot 12 with the saline solution 20 into the left nostril 22a so that it forms a comfortable seal.

The user then places the v-shaped recessed lip 14b of the container 14 against the right nostril 22b using their right hand. This creates a seal between the lip 14b of the container 14 and the right nostril 22b. This prevents the solution and mucus from running down the cheeks and onto clothing. Typically, the user should breathe with their mouth open in order to breathe properly.

Now referring to FIG. 6B, an example of the next step for using a portable sinus cleansing kit 10 in accordance with one embodiment of the present invention is presented. As shown, the user rotates their head so that the left nostril 22a is above the right nostril 22b. During this process, the forehead should remain higher than the chin.

Next, the user raises the handle of the pot 12 so that the solution 20 enters the left nostril 22a. Solution 20 will begin to drain out of the right nostril 22b and into the container 14. Typically, the user does not inhale or snort the solution 20 into the nose and the user should continue to breathe out of their mouth. When the pot 12 is empty, the user exhales through both the left nostril 22a and the right nostril 22b to clear them of excess mucus and solution 20. The mucus and the saline solution 20 can be placed in the container 14. In addition, the user can blow their nose 22 with a tissue. This procedure should be repeated on either side to thoroughly clean the nasal cavity. After the user completes cleaning the nasal cavity, the user should clean all parts of the kit 10 after each use.

After removing mucus and other irritants from the nasal cavity, the user removes mucus from their mouth and throat. Inflammation can still exist even though the nasal cavity has been cleaned out. The mouth is the most common route of entry for organisms other than the nose that cause cold and flu symptoms. As such, the kit 10 can also be used to cleanse the mouth and throat of the user

To cleanse the mouth and throat of the user, the user fills the container lid 14a with lukewarm water. In one embodiment of the present invention, the container lid 14a is filled up to the 8 oz mark with water. The user then empties half the dry salt packet 18b into the container lid 14a. The user stirs the contents of the container lid 14a with a spoon 16 until the contents have been completely dissolved forming the salt solution. One skilled in the art would appreciate that the concentration of the salt solution depends on the amount of salt and the amount of water.

To remove mucus or any other irritants in the mouth and throat, the user pours the salt solution into their mouth. The user slides the salt solution quickly over their tongue. In turn, the user throws their head back and lets the solution rest inside the mouth right above the epiglottis. Typically, the user will gargle the salt solution for about 45 sections. The user can then discharge the solution into the container 14. Alternatively, the user can discharge the solution into a sink. In general, the user will gargle the salt solution three times a day to flush away the unwanted mucus, bacteria, and other viruses of the throat.

The user can repeat the steps for cleaning out the nasal cavity and the mouth and throat as many times as they desire. After the user is finished, the user cleans the kit 10 and easily packs it away.

While the present invention has been described with regards to particular embodiments, it is recognized that additional variations of the present invention may be devised without departing from the inventive concept.

What is claimed is:

1. A mucus cleansing kit comprising:
a container lid;
at least one or more packets of dry saline;
a spoon for mixing the dry saline from the at least one or more packets with water to produce a saline solution;
a pot for holding the saline solution, wherein the saline solution from the pot is poured into a nostril of a user cleansing a nasal cavity of mucus;
at least one or more packets of dry salt, wherein the spoon is used for mixing the dry salt from the at least one or more packets with water to produce a salt solution, the salt solution poured into the mouth of the user cleansing a mouth and throat of mucus; and
a container for collecting the saline solution, salt solution, and mucus.

2. The mucus cleansing kit of claim 1, wherein the saline solution comprises a sterile solution of sodium chloride in water.

3. The mucus cleansing kit of claim 1, wherein the water to produce the saline solution and salt solution is warm water.

4. The mucus cleansing kit of claim 1, wherein the container lid comprises a plurality of measurement guides for displaying an amount of liquid inside the container lid.

5. The mucus cleansing kit of claim 4, wherein the plurality of measurement guides includes a 4 oz. marker.

6. The mucus cleansing kit of claim 4, wherein the plurality of measurement guides includes a 8 oz. marker.

7. The mucus cleansing kit of claim 1, wherein the container and the container lid house the spoon, the pot, the at least one or more packets of dry saline, and the at least one or more packets of dry salt.

8. A method of cleansing mucus comprising:
removing mucus from a nasal cavity by pouring a saline solution into a nostril of a user; and
removing mucus from a throat by pouring a salt solution into a mouth of the user.

9. The method of claim 8, further comprising producing the saline solution by mixing dry saline with water.

10. The method of claim 8, further comprising producing the salt solution by mixing dry salt with water.

11. The method of claim 8, wherein cleansing mucus from the nasal cavity by pouring the saline solution into the nostril of the user comprises:
inserting a spout of a pot into a first nostril of the user;
rotating a head of the user so that the first nostril with the spout is above a second nostril;
placing a container under the second nostril; and
pouring the saline solution into the first nostril of the user, wherein the saline solution and the mucus empties out of the second nostril into the container.

12. The method of claim 11, further comprising:
inserting the spout of the pot into the second nostril of the user;
rotating the head of the user so that the second nostril with the spout is above the first nostril;
placing the container under the first nostril; and
pouring the saline solution into the second nostril of the user, wherein the saline solution and the mucus empties out of the first nostril into the container.

13. The method of claim 8, wherein cleansing mucus from the throat by pouring the salt solution into the mouth of the user comprises gargling the salt solution.

14. The method of claim 8, wherein cleansing mucus from the nasal cavity by pouring the saline solution into the nostril of the user comprises creating a seal between the nostril and a pot.

15. A method for cleansing mucus with a kit having at least one or more packets of dry saline, a pot, a spoon, a container, and a container lid, the method comprising:
mixing the at least one or more packets of dry saline and water with the spoon;
inserting a spout of the pot into a first nostril of the user;
rotating the head of the user so that the first nostril with the spout is above a second nostril;
placing the container under the second nostril; and
pouring the saline solution into the first nostril of the user, wherein the saline solution and the mucus empties out of the second nostril into the container.

16. The method of claim 15, further comprises gargling salt solution to remove mucus from a mouth and throat of the user, wherein the salt solution is made from at least one or more packets of dry salt and water.

17. The method of claim 16, further comprises repeating the method to remove all mucus from a nasal cavity, the mouth, and the throat of the user.

18. The method of claim 15, wherein inserting the spout of the pot into a first nostril of the user comprises creating a seal between the first nostril and the pot.

19. The method of claim 15, further comprising packing the cleansing kit by placing the at least one or more packets of dry saline, the pot, and the spoon within the container and the container lid.

20. The method of claim 15, wherein mixing the at least one or more packets of dry saline and water with the spoon comprises heating the water for dissolving the dry saline.
